(19) United States
(12) Patent Application Publication
(10) Pub. No.: US 2020/0098478 A1
(43) Pub. Date: Mar. 26, 2020

(54) DEVICE, METHOD AND PROGRAM FOR EVALUATING SENSITIVITY OF BLOOD PRESSURE TO Na/K RATIO

(71) Applicant: OMRON HEALTHCARE Co., Ltd., Muko-shi (JP)

(72) Inventors: Ken MIYAGAWA, Kyoto (JP); Masakazu TSUTSUMI, Kyoto (JP)

(73) Assignee: OMRON HEALTHCARE Co., Ltd., Muko-shi (JP)

(21) Appl. No.: 16/695,773

(22) Filed: Nov. 26, 2019

Related U.S. Application Data
(63) Continuation of application No. PCT/JP2018/021003, filed on May 31, 2018.

(30) Foreign Application Priority Data
Jun. 16, 2017 (JP) 2017-118987

(51) Int. Cl. G16H 50/20; G16H 10/60; G16H 20/60; A61B 5/022; A61B 5/20; A61B 5/00; G01N 33/84; G01N 33/493

(52) U.S. Cl. CPC G16H 50/20; G16H 10/60; G16H 20/60; A61B 5/022; G01N 33/493; A61B 5/207; A61B 5/742; G01N 33/84; A61B 5/201

(57) ABSTRACT

A device inputs blood pressure data and urinary Na/K ratio data while associating the blood pressure data and the urinary Na/K ratio data with each other. A scatter diagram representing data points decided by the Na/K ratio data and the blood pressure data associated with each other is produced on a plane formed by a first coordinate axis representing the Na/K ratio and a second coordinate axis representing the blood pressure, and sensitivity type of a subject is determined as one of predetermined four types existing actually according to a distribution of the data points in the scatter diagram. Information representing a determination result is output.

### CROSS-REFERENCE TO RELATED APPLICATIONS

This is a continuation application of International Application No. PCT/JP2018/021003, with an International filing date of May 31, 2018, which claims priority of Japanese Patent Application No. 2017-118987 filed on Jun. 16, 2017, the entire content of which is hereby incorporated by reference.

### FIELD

The present invention relates to a device and a method for evaluating Na/K ratio sensitivity of a blood pressure, namely, sensitivity of a blood pressure to a ratio of a sodium amount and a potassium amount in urine or intake food. The present invention also relates to a program for causing a computer to perform the method.

### BACKGROUND

It is known that a significant positive correlation exists between a blood pressure and a urinary Na/K ratio (for example, see Non-Patent Literature 1 below). Ratios of amounts of sodium and potassium taken in by the meal and excreted in the urine by human are 86% and 77%, respectively (see Non-patent Literature 2 below). Based on the knowledge, a dietary cure is performed for a hypertension patient in order to restrict a ratio of sodium and potassium (Na/K ratio) in the intake food.

Non-patent Literature 1: Naosuke Sasaki, “Relation of urinary findings, especially sodium potassium ratio, to blood pressure levels in the northeast of Japan”, “Medicine and Biology”, 39 (6), P 182-187, June 1956

Non-patent Literature 2: “Nutritional Epidemiology” written by Walter Willett, translated by Heizo Tanaka; 2nd edition; DAI-ICHI SHUPPAN Co., Ltd.; May 2003

### SUMMARY

Until now, in medical practice, a correlation between a blood pressure (particularly systolic blood pressure (SBP)) and a urinary Na/K ratio is approximated by one straight line L0 as illustrated in FIG. 9 (In FIG. 9, a data point decided by the systolic blood pressure data and the corresponding urinary Na/K ratio is indicated by a mark “0”. The same applies to FIGS. 7A to 7D (to be described later.)). For example, guidance for restricting the Na/K ratio in intake food is performed such that the systolic blood pressure falls below a hypertension reference value UL (=135 mmHg) of a home blood pressure stipulated in Guidelines for the Management of Hypertension 2014 (Japanese Society of Hypertension). In an example of FIG. 9, guidance is performed such that the urinary Na/K ratio is less than or equal to 3.5, and therefore the Na/K ratio in intake food is less than or equal to about 3.1 by conversion.

The present inventor acquired the systolic blood pressure data and the urinary Na/K ratio data a plurality of times over a certain period for a plurality of subjects while associating the systolic blood pressure data and the urinary Na/K ratio data with each other for each subject, and performed analysis by producing a scatter diagram on a plane 99 formed by a horizontal axis (x-axis) representing the urinary Na/K ratio and a vertical axis (y-axis) representing the systolic blood pressure as illustrated in FIGS. 7A to 7D. As a result, it was found that four types exists actually in the correlation between the systolic blood pressure and the urinary Na/K ratio. The four types are
as illustrated in FIG. 7A, a type in which, on the plane 99, the blood pressure with respect to the Na/K ratio changes along one straight line L1 over a range from a lower limit portion DL1 to an upper limit portion DU1 where the data points are distributed (this is referred to as a “first type”),
as illustrated in FIG. 7B, a type in which, on the plane 99, the blood pressure with respect to the Na/K ratio changes over the range from a lower limit portion DL2 to an upper limit portion DU2 where the data points are distributed while being curved more convexly (downward convex) than one straight line L2 connecting the lower limit portion DL2 and the upper limit portion DU2 with respect to the horizontal axis (x-axis) (this is referred to as a “second type”. A curve that approximates the above distribution indicated by symbol C2),
as illustrated in FIG. 7C, a type in which, on the plane 99, the blood pressure with respect to the Na/K ratio changes over the range from a lower limit portion DL3 to an upper limit portion DU3 where the data points are distributed while being curved more concavely (upward convex) than one straight line L3 connecting the lower limit portion DL3 and the upper limit portion DU3 with respect to the horizontal axis (x-axis) (this is referred to as a “third type”. A curve that approximates the above distribution indicated by symbol C3), and
as illustrated in FIG. 7D, a type having no correlation between the Na/K ratio and the blood pressure (this is referred to as a “fourth type”).

Thus, if it is determined for each subject which one of the four types (this is appropriately referred to as “sensitivity type”) the correlation between the systolic blood pressure and the urinary Na/K ratio of the subject belongs to, and if this determination result is reflected in the guidance to the hypertension patient or the like, then the accuracy of the guidance will be increased.

Accordingly, an object of the present invention is to provide a device that evaluates the Na/K ratio sensitivity of the blood pressure and can determine the sensitivity type of the subject. Another object of the present invention is to provide a method for evaluating the Na/K ratio sensitivity of the blood pressure and being able to determine the sensitivity type of the subject. Still another object of the present invention is to provide a program causing a computer to perform the method.

In order to achieve the above object, a device of the present disclosure is a device that evaluates Na/K ratio sensitivity of a blood pressure, the device comprising:

a data input unit that inputs blood pressure data and urinary Na/K ratio data measured a plurality of times over a certain period with respect to a certain subject while associating the blood pressure data and the urinary Na/K ratio data with each other;

a type determination unit that produces a scatter diagram representing data points decided by the Na/K ratio data and the blood pressure data associated with each other on a plane formed by a first coordinate axis representing a Na/K ratio and a second coordinate axis representing a blood pressure, and determines sensitivity type of the subject as one of predetermined four types existing actually according to a distribution of the data points in the scatter diagram; and

an output unit that outputs information representing a determination result of the type determination unit.

Typically, the “first coordinate axis” is the horizontal axis (x-axis) and the “second coordinate axis” is the vertical axis (y-axis), and may be reversed.

“Information indicating the determination result” may widely include information relating to the Na/K ratio sensitivity of the blood pressure, such as a name indicating the determined sensitivity type, the image representing the scatter diagram, and advice according to each of the four types.

In another aspect, a method of the present disclosure is a method for assessing Na/K ratio sensitivity of a blood pressure, the method comprising:

inputting blood pressure data and urinary Na/K ratio data measured a plurality of times over a certain period with respect to a certain subject while associating the blood pressure data and the urinary Na/K ratio data with each other;

producing a scatter diagram representing data points decided by the Na/K ratio data and the blood pressure data associated with each other on a plane formed by a first coordinate axis representing a Na/K ratio and a second coordinate axis representing a blood pressure, and determining sensitivity type of the subject as one of predetermined four types existing actually according to a distribution of data points in the scatter diagram; and

outputting information representing a determination result.

In another aspect, a program of the present disclosure is a program causing a computer to perform the method.

### BRIEF DESCRIPTION OF DRAWINGS

The present invention will become more fully understood from the detailed description given hereinbelow and the accompanying drawings which are given by way of illustration only, and thus are not limitative of the present invention, and wherein:

FIG. 1 is a view illustrating a block configuration of a device that evaluates Na/K ratio sensitivity of a blood pressure according to an embodiment of the present invention.

FIG. 2 is a view illustrating an operation flow of a method for evaluating the Na/K ratio sensitivity of the blood pressure of the embodiment, the method being performed by the device in FIG. 1.

FIG. 3 is a view illustrating a detailed flow for determining (type determination) a sensitivity type of a subject as one of four types existing actually, the determination being included in the operation flow in FIG. 2.

FIG. 4A is a scatter diagram illustrating a distribution of data points decided by urinary Na/K ratio data and blood pressure data when the sensitivity type of the subject is a first type (straight line type).

FIG. 4B is a scatter diagram illustrating the distribution of the data points decided by the urinary Na/K ratio data and the blood pressure data when the sensitivity type of the subject is a second type (downward convex type).

FIG. 4C is a scatter diagram illustrating the distribution of the data points decided by the urinary Na/K ratio data and the blood pressure data when the sensitivity type of the subject is a third type (upward convex type).

FIG. 4D is a scatter diagram illustrating the distribution of the data points decided by the urinary Na/K ratio data and the blood pressure data when the sensitivity type of the subject is a fourth type (no correlation type).

FIG. 5A is a view illustrating a display example output when the sensitivity type of the subject is the first type (straight line type).

FIG. 5B is a view illustrating a display example output when the sensitivity type of the subject is the second type (downward convex type).

FIG. 5C is a view illustrating a display example output when the sensitivity type of the subject is the third type (upward convex type).

FIG. 5D is a view illustrating a display example output when the sensitivity type of the subject is the fourth type (no correlation type).

FIG. 6A is a view illustrating a processing flow for setting a time difference associating measured blood pressure data with measured Na/K ratio data for each subject.

FIG. 6B is a view illustrating an example in which the measured blood pressure data and the measured Na/K ratio data change with time.

FIG. 7A is a scatter diagram illustrating the distribution of the data points decided by the urinary Na/K ratio data and the blood pressure data of the first type (straight line type) in the four types existing actually as a correlation between a systolic blood pressure and the urinary Na/K ratio.

FIG. 7B is a scatter diagram illustrating the distribution of the data points decided by the urinary Na/K ratio data and the blood pressure data of the second type (downward convex type) in the four types existing actually as a correlation between a systolic blood pressure and the urinary Na/K ratio.

FIG. 7C is a scatter diagram illustrating the distribution of the data points decided by the urinary Na/K ratio data and the blood pressure data of the third type (upward convex type) in the four types existing actually as a correlation between a systolic blood pressure and the urinary Na/K ratio.

FIG. 7D is a scatter diagram illustrating the distribution of the data points decided by the urinary Na/K ratio data and the blood pressure data of the fourth type (no correlation type) in the four types existing actually as a correlation between a systolic blood pressure and the urinary Na/K ratio.

FIG. 8 is a view illustrating an example in which a horizontal axis with a scale indicating the Na/K ratio in intake food is added in parallel to the scatter diagram in FIG. 5B.

FIG. 9 is a view illustrating a conventional way to evaluate the correlation between the blood pressure and the urinary Na/K ratio.

### DETAILED DESCRIPTION

Hereinafter, an embodiment of the present invention will be described in detail with reference to the drawings.

### (Schematic Configuration of Device)

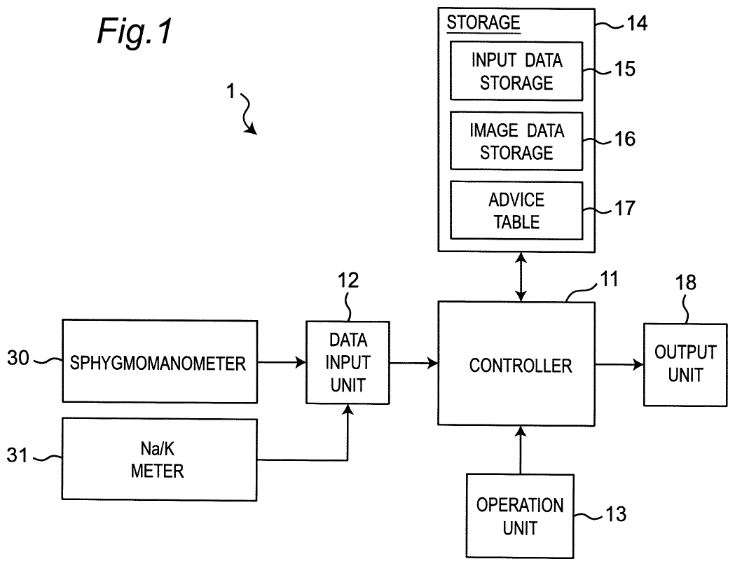

FIG. 1 illustrates a block configuration of a device 1 that evaluates Na/K ratio sensitivity of a blood pressure according to an embodiment of the present invention.

The device 1 includes a controller 11, a data input unit 12, an operation unit 13, a storage 14, and an output unit 18. In this example, a sphygmomanometer 30 that measures blood pressure data and a Na/K meter 31 serving as measuring instrument that measures urinary Na/K ratio data are connected to the data input unit 12 by wireless communication.

The sphygmomanometer 30 measures the blood pressure data of a subject every time a measurement instruction is received from the subject (spot measurement). In this example, the sphygmomanometer 30 is a commercially available sphygmomanometer (in this example, “OMRON Upper Arm Sphygmomanometer HEM-7511T” manufactured by OMRON Healthcare Co., Ltd.).

The Na/K meter 31 measures the urinary Na/K ratio at any time every time the measurement instruction is received from the subject (spot measurement). In this example, the Na/K meter 31 is a commercially available measuring instrument (in this example, “OMRON Na⁺K⁺scan HEU-001F” manufactured by OMRON Healthcare Co., Ltd.).

The controller 11 includes a CPU (Central Processing Unit) operated by software (computer program) and an auxiliary circuit of the CPU, and executes various pieces of processing (to be described later) according to a program and data stored in the storage 14.

The operation unit 13 includes a known keyboard and mouse, and functions to input a command and various pieces of information from a user. Examples of the command include a command to instruct input or start of processing and a command to instruct recording of image data.

The data input unit 12 is constructed with a known input interface, and sequentially inputs the blood pressure data measured by the sphygmomanometer 30 a plurality of times over a certain period and the urinary Na/K ratio data measured by the Na/K meter 31 a plurality of times over substantially the same period, respectively in real time for each measurement with respect to a certain subject by the processing of the controller 11, in this example. The input blood pressure data and urinary Na/K ratio data are associated with each other through a 12-hour time difference by the processing of the controller 11 (to be described later), and stored in the input data storage 15 together with date and time of each measurement.

In this example, the storage 14 includes the input data storage 15 constructed with a RAM (Random Access Memory) used as a work area necessary for execution of a program using the controller 11, a hard disk drive in which a basic program executed by the controller 11 is stored, and an EEPROM (electrically rewritable nonvolatile memory) in which data is non-temporarily stored, an image data storage 16, and an advice table 17.

The input data storage 15 stores the blood pressure data and the urinary Na/K ratio data measured a plurality of times over a certain period with respect to a certain subject together with the date and time of each measurement while associating the blood pressure data and the urinary Na/K ratio data with each other. When the input data about the plurality of subjects are stored, an identification number (ID) is attached for each subject, and the input data is stored for each subject.

The image data storage 16 stores the output image data produced by processing (to be described later) of the controller 11.

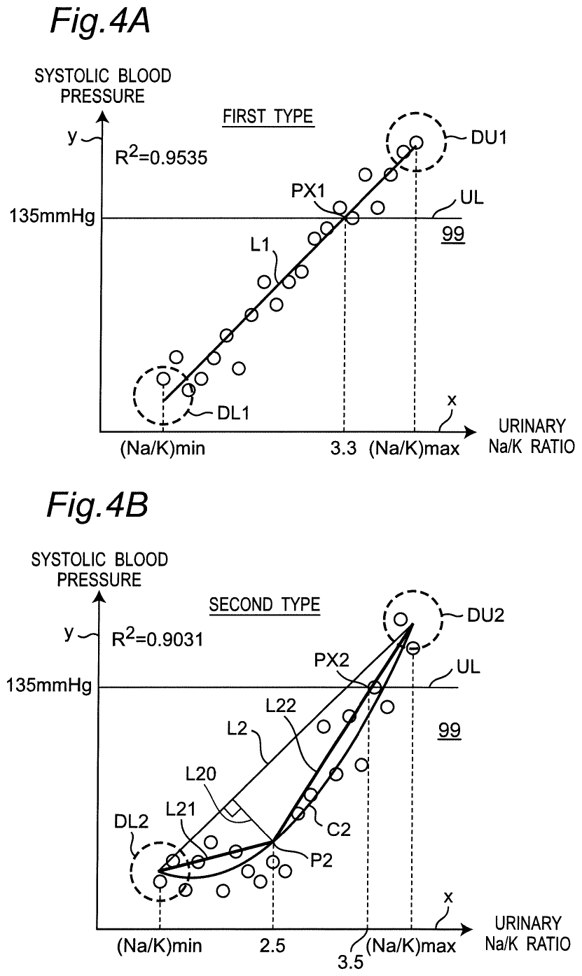

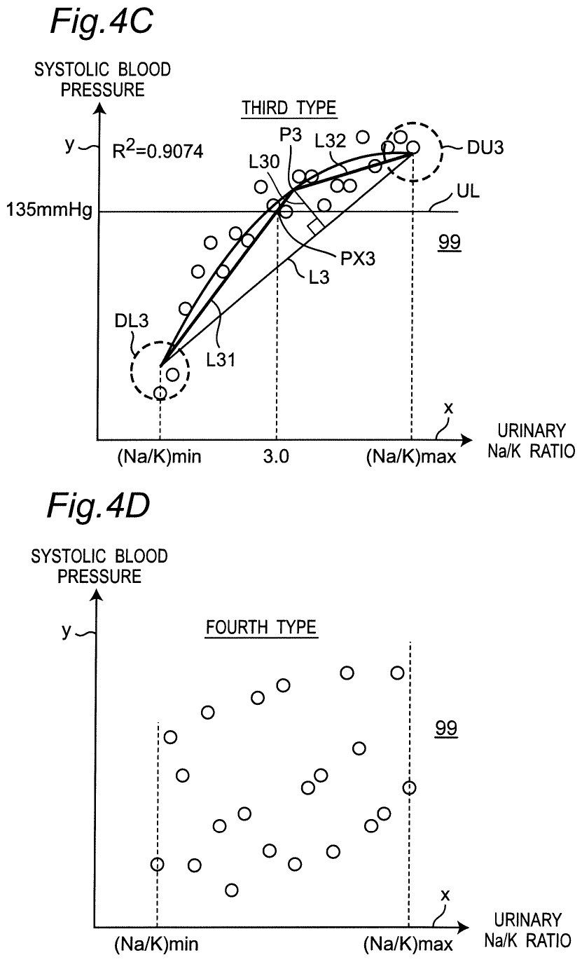

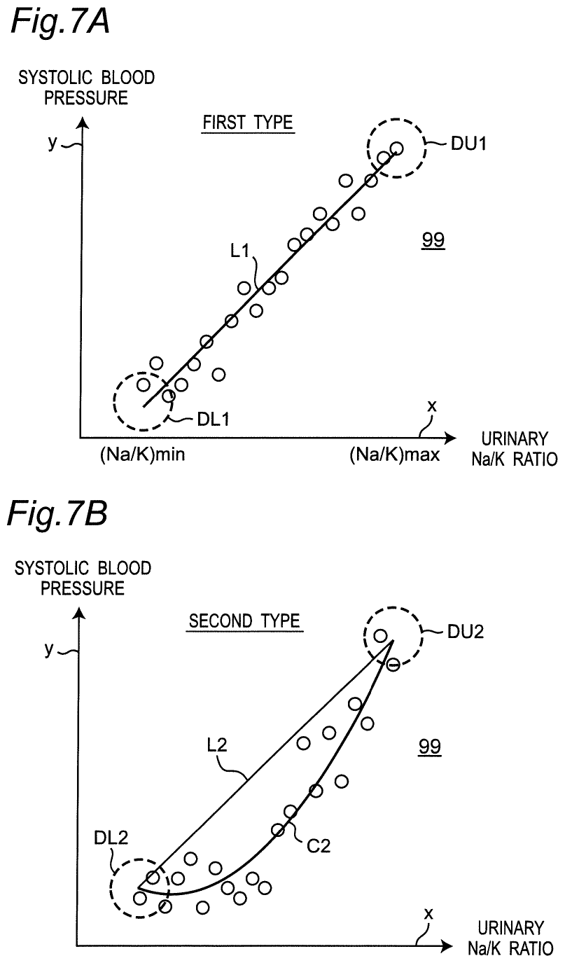

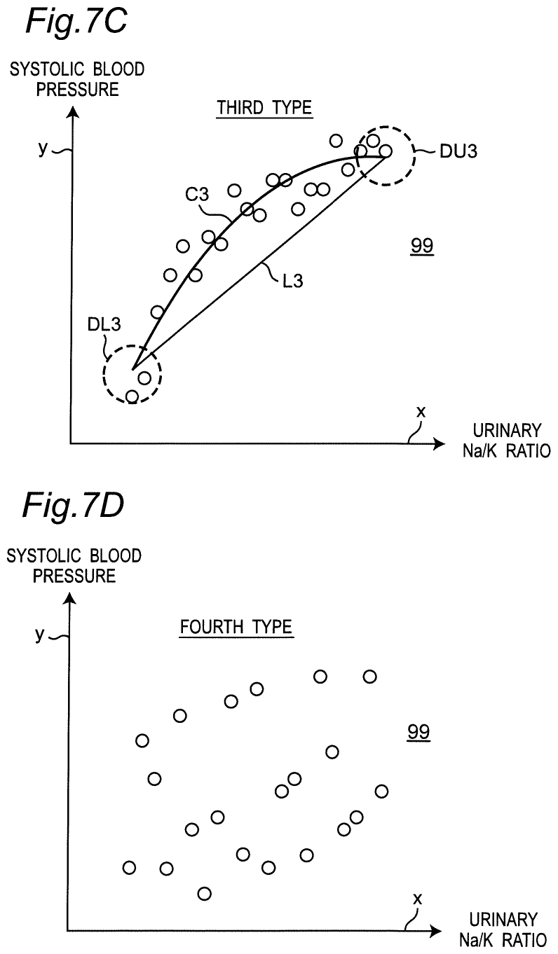

The advice table 17 previously stores sentence examples of advice according to four types (first to fourth types) representing the correlation between a systolic blood pressure and the urinary Na/K ratio as illustrated in FIGS. 7A to 7D described above (or FIGS. 4A to 4D (to be described later)). A sentence example of the advice will be described later.

In this example, the output unit 18 in FIG. 1 is constructed with an LCD (liquid crystal display element), and displays various pieces of information such as processing result of the controller 11, in this example particularly information indicating the determination result on the display screen. The output unit 18 may include a printer (driver), perform print out on paper, and output a processing result.

### (Method for Evaluating Na/K Ratio Sensitivity of Blood Pressure)

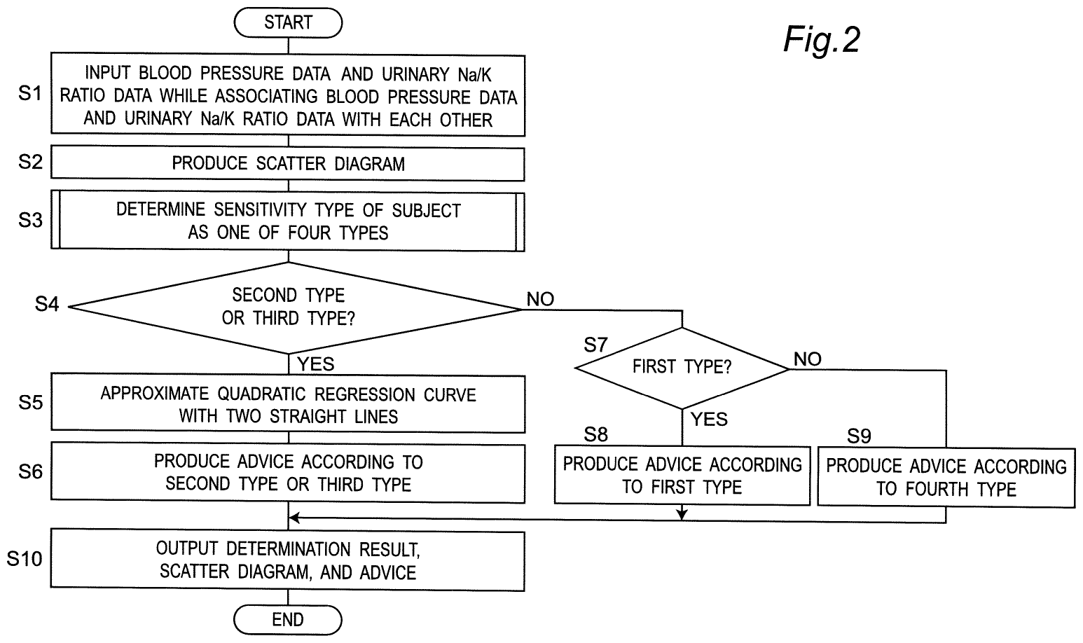

Under control of the controller 11, the device 1 operates as a whole according to the processing flow in FIG. 2.

### (1) Data Input

As illustrated in step S1 of FIG. 2, via the data input unit 12, the controller 11 sequentially inputs the blood pressure data measured by the sphygmomanometer 30 a plurality of times over a certain period and the urinary Na/K ratio data measured by the Na/K meter 31 a plurality of times over substantially the same period as the blood pressure data, respectively in real time for each measurement with respect to a certain subject by the processing of the controller 11, in this example. In this example, the input blood pressure data and urinary Na/K ratio data are associated with each other through the 12-hour time difference by the processing of the controller 11. That is, blood pressure data input at a certain time is associated with the urinary Na/K ratio data input 12 hours after that time. The reason for the association through the 12-hour time difference is that, generally, an influence of sodium and potassium taken in by a human through a meal appears first in the blood pressure, and appears in the urinary Na/K ratio approximately half a day later (12 hours). The controller 11 stores the blood pressure data and the urinary Na/K ratio data in the input data storage 15 together with the date and time of each measurement while associating the blood pressure data and the urinary Na/K ratio data with each other.

In order to accurately evaluate the Na/K ratio sensitivity of the blood pressure, desirably a period in which the subject measures the blood pressure data and the urinary Na/K ratio data is greater than or equal to several days, and the number of measurement times is greater than or equal to 20.

Subsequently, as illustrated in step S2, the controller 11 produces a scatter diagram in which data points (indicated by a mark “o”) decided by the Na/K ratio data and the blood pressure data associated with each other is represented on a plane 99 formed by a horizontal axis (x axis) as a first coordinate axis representing the Na/K ratio and a vertical axis (y-axis) as a second coordinate axis representing the blood pressure as illustrated in FIGS. 4A to 4D. The produced scatter diagram is stored in the image data storage 16.

### (2) Type Determination

As illustrated in step S3 of FIG. 2, the controller 11 functions as a type determination unit, and determines a sensitivity type of the subject as one of predetermined four types (first to fourth types) existing actually according to a distribution of data points in the scatter diagram (type determination).

In the same as described with reference to FIGS. 7A to 7D, the four types are
as illustrated in FIG. 4A, the first type in which, on the plane 99, the blood pressure with respect to the Na/K ratio changes along one straight line L1 over the range from a lower limit portion DL1 to an upper limit portion DU1 where the data points are distributed,
as illustrated in FIG. 4B, the second type in which, on the plane 99, the blood pressure with respect to the Na/K ratio changes over the range from a lower limit portion DL2 to an upper limit portion DU2 where the data points are distributed while being curved more convexly (downward convex) than one straight line L2 connecting the lower limit portion DL2 and the upper limit portion DU2 with respect to the horizontal axis (x-axis),
as illustrated in FIG. 4C, the third type in which, on the plane 99, the blood pressure with respect to the Na/K ratio changes over the range from a lower limit portion DL3 to an upper limit portion DU3 where the data points are distributed while being curved more concavely (upward convex) than one straight line L3 connecting the lower limit portion DL3 and the upper limit portion DU3 with respect to the horizontal axis (x-axis), and
as illustrated in FIG. 4D, the fourth type having no correlation between the Na/K ratio and the blood pressure. (Na/K) min and (Na/K) max indicated on each horizontal axis (x-axis) represent a minimum value and a maximum value of the Na/K ratio data, respectively.

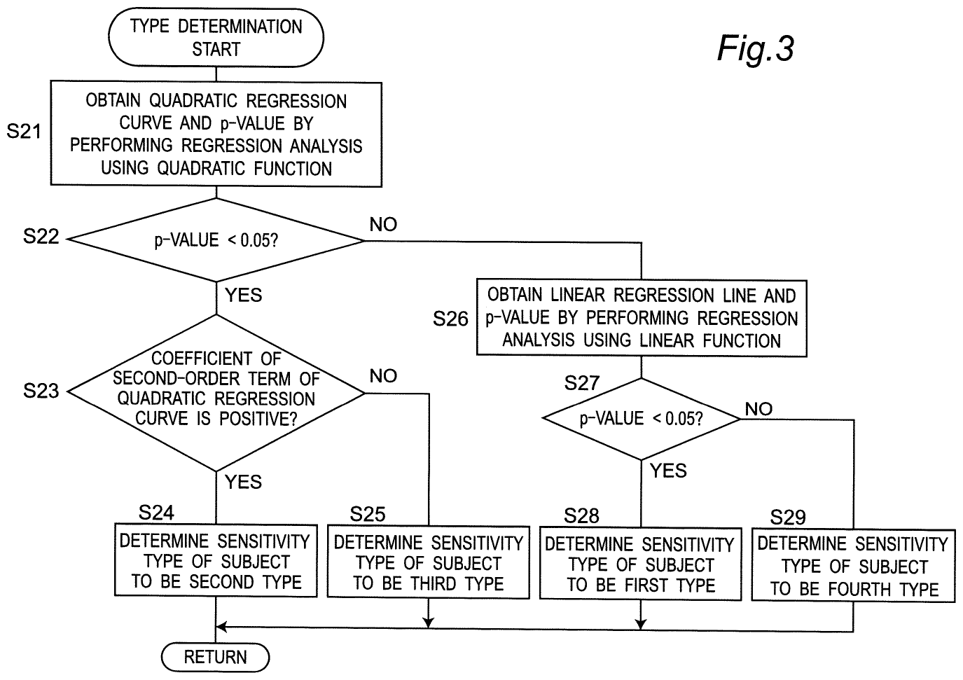

FIG. 3 illustrates a specific flow of the type determination.

As illustrated in step S21 of FIG. 3, a regression analysis is performed on the distribution using a quadratic function to obtain a quadratic regression curve and a p-value indicating significance. In this example, a regression analysis is performed by a least squares method using a quadratic function y=ax²+bx+c (where a, b, c represent a coefficient of a second-order term, a coefficient of a first-order term, and a constant, respectively) to obtain a quadratic regression curve that approximates the above distribution (that is, a, b, and c are obtained). A p-value indicating the significance of the regression analysis is obtained.

Subsequently, whether the p-value is less than a predetermined first threshold (in this example, 0.05) is determined as illustrated in step S22. The determination that the sensitivity type of the subject belongs to the group of the second type and the third type is made when the p-value is less than the first threshold (=0.05) (YES in step S22). In this case, the distribution is one along a curve C2 that is curved convexly downward as illustrated in FIG. 4B or one along a curve C3 that is curved convexly upward as illustrated in FIG. 4C. For this reason, the processing proceeds to step S23 in FIG. 3 to determine whether the sensitivity type of the subject is the second type or the third type according to the sign of the coefficient a of the second-order term of the quadratic regression curve. That is, the determination that the sensitivity type of the subject is the second type (that is, the downward convex type) as illustrated in FIG. 4B is made (step S24) when the coefficient a is greater than zero (YES in step S23). On the other hand, the determination that the sensitivity type of the subject is the third type (that is, the upward convex type) as illustrated in FIG. 4C (step S25) is made when the coefficient a is less than zero (NO in step S23).

On the other hand, the determination that the sensitivity type of the subject belongs to the group of the first type and the fourth type is made when the p-value is greater than or equal to the first threshold (=0.05) in step S22 of FIG. 3 (NO in step S22). In this case, the processing proceeds to step S26, and a regression analysis is further performed on the distribution using a linear function to obtain a linear regression line and the p-value indicating the significance. In this example, the regression analysis is performed by the least squares method using a linear function y=dx+e (where d, e represent a coefficient of a first-order term and a constant, respectively) to obtain the linear regression line L1 that approximates the above distribution (that is, d and e are obtained). A p-value indicating the significance of the regression analysis is obtained.

Subsequently, whether the p-value is less than a predetermined second threshold (in this example, 0.05) is determined as illustrated in step S27. The determination that the sensitivity type of the subject is the first type (that is, the straight line type) as illustrated in FIG. 4A is made (step S28) when the p-value is less than the second threshold (=0.05) (YES in step S27). On the other hand, in step S27 of FIG. 3, the determination that the sensitivity type of the subject is the fourth type (that is, the no correlation type) as illustrated in FIG. 4D is made (step S29) when the p-value is greater than or equal to the first threshold (=0.05) (NO in step S27).

In this way, the type determination is made. According to this determination way, the sensitivity type of the subject can accurately be determined as one of the sensitivity types existing actually by simple processing. The sensitivity type of the subject obtained as a result of this type determination is stored in the storage 14. A determination coefficient R²=0.9535 in the example of FIG. 4A, the determination coefficient R²=0.9031 in the example of FIG. 4B, and the determination coefficient R²=0.9074 in the example of FIG. 4C. Then, the processing returns to step S4 of the main flow (FIG. 2).

### (3) Two-Line Approximation

When the sensitivity type of the subject obtained as the result of the type determination is the second type or the third type (YES in step S4 of FIG. 2), the processing proceeds to step S5, and the controller 11 acts as a two-line approximation unit to perform processing of approximating the quadratic regression curve C2 or C3 with two straight lines on the plane 99 in FIG. 4B or 4C.

For example, when the sensitivity type of the subject is the second type, a first straight line L21 and a second straight line L22 that pass through the lower limit portion DL2 and the upper limit portion DU2, respectively and are connected and bent to each other at a certain transition point P2 along the quadratic regression curve C2 are obtained so as to approximate the quadratic regression curve C2 on the plane 99 in FIG. 4B. In this example, the transition point P2 is such one at which the quadratic regression curve C2 is separated farthest from one straight line L2 connecting the lower limit portion DL2 and the upper limit portion DU2. Actually, a perpendicular line L20 with respect to the straight line L2 is drawn from the straight line L2 to the curve C2, and a point on the curve C2 where the length of the perpendicular line L20 is maximum is obtained as the transition point P2. A straight line passing through the lower limit portion DL2 (particularly, a point on the straight line L2 corresponding to the minimum value (Na/K) min of the Na/K ratio data) and the transition point P2 is obtained as the first straight line L21. A straight line passing through the transition point P2 and the upper limit portion DU2 (particularly, a point on the straight line L2 corresponding to the maximum value (Na/K) max of the Na/K ratio data) is obtained as the second straight line L22. The obtained first straight line L21 and second straight line L22 are stored while overwritten on the scatter diagram in FIG. 4B stored in the image data storage 16.

In this case, the range from the lower limit portion DL2 to the transition point P2 can be approximated by the first straight line L21, and the range from the transition point P2 to the upper limit portion DU2 can be approximated by the second straight line L22. Consequently, the accuracy of the approximation can be improved as compared with the case where the correlation between the blood pressure and the urinary Na/K ratio is simply approximated by one straight line L2. The user easily recognizes a slope of the correlation between the blood pressure and the urinary Na/K ratio while dividing the slope of the correlation between the blood pressure and the urinary Na/K ratio in the range from the lower limit portion DL2 to the transition point P2 and the range from the transition point P2 to the upper limit portion DU2.

Similarly, when the sensitivity type of the subject is the third type, a first straight line L31 and a second straight line L32 that pass through the lower limit portion DL3 and the upper limit portion DU3, respectively and are connected and bent to each other at a certain transition point P3 along the quadratic regression curve C3 are obtained so as to approximate the quadratic regression curve C3 on the plane 99 in FIG. 4C. In this example, the transition point P3 is such one at which the quadratic regression curve C3 is separated farthest from one straight line L3 connecting the lower limit portion DL3 and the upper limit portion DU3. Actually, a perpendicular line L30 with respect to the straight line L3 is drawn from the straight line L3 to the curve C3, and a point on the curve C3 where the length of the perpendicular line L30 is maximum is obtained as the transition point P3. A straight line passing through the lower limit portion DL3 (particularly, a point on the straight line L3 corresponding to the minimum value (Na/K) min of the Na/K ratio data) and the transition point P3 is obtained as the first straight line L31. A straight line passing through the transition point P3 and the upper limit portion DU3 (particularly, a point on the straight line L3 corresponding to the maximum value (Na/K) max of the Na/K ratio data) is obtained as the second straight line L32. The obtained first straight line L31 and second straight line L32 are stored while overwritten on the scatter diagram in FIG. 4C stored in the image data storage 16.

In this case, the range from the lower limit portion DL3 to the transition point P3 can be approximated by the first straight line L31, and the range from the transition point P3 to the upper limit portion DU3 can be approximated by the second straight line L32. Consequently, the accuracy of the approximation can be improved as compared with the case where the correlation between the blood pressure and the urinary Na/K ratio is simply approximated by one straight line L3. The user easily recognizes a slope of the correlation between the blood pressure and the urinary Na/K ratio while dividing the slope of the correlation between the blood pressure and the urinary Na/K ratio in the range from the lower limit portion DL3 to the transition point P3 and the range from the transition point P3 to the upper limit portion DU3.

### (4) Production of Advice

Thereafter, the processing proceeds to step S6 in FIG. 2, and the controller 11 acts as an advice production unit to produce advice according to the second type or the third type.

When the sensitivity type of the subject obtained as the result of the type determination is the first type (NO in step S4 and YES in step S7 of FIG. 2), the processing proceeds to step S8, and the controller 11 acts as the advice production unit to the advice according to the first type.

When the sensitivity type of the subject obtained as the result of the type determination is the fourth type (NO in step S4 and NO in step S7 of FIG. 2), the processing proceeds to step S9, and the controller 11 acts as the advice production unit to the advice according to the fourth type.

Specifically, the advice for the four types (first to fourth types) is produced as follows.

For example, it is assumed that a sentence example of advice for the first type stored in the advice table 17 is such one shown in Table 1.

TABLE 1
Sentence example of advice for first type

Your blood pressure became 135 mmHg (a hypertension reference value
of a home blood pressure) when a Na/K ratio was “AAA”.
In order to keep your blood pressure within the reference value, you
should not take the meal having a Na/K ratio of “AAA” or more.
The meal having a Na/K ratio of “AAA” was of “□/□□” (date).

At this point, a numerical value of Na/K ratio (two significant digits) is applied to a field of “AAA” by the processing (to be described later). In a field of “□/□□” column, a numerical value of month/day values is applied.

When the sensitivity type of the subject is the first type to illustrate the distribution of the data points in FIG. 4A, an x-coordinate (Na/K ratio coordinate) of an intersection point PX1 of a linear regression line L1 and a hypertension reference value UL (=135 mmHg) is 3.3. Thus, the controller 11 reads out the sentence example in Table 1 from the advice table 17, applies a value according to the distribution of the data points to the field of the “AAA” of the sentence example, in this example, applies the value of 3.3 of the x-coordinate of the intersection point PX1 to the field of the “AAA”, and produces the advice in Table 1-A.

TABLE 1-A
Advice for first type

Your blood pressure became 135 mmHg (the hypertension reference value
of the home blood pressure) when a Na/K ratio was 3.3.
Do not take the meal having a Na/K ratio of 3.3 or more in order to keep
your blood pressure within the reference value.
The meal having a Na/K ratio of 3.3 was of 5/08 (date).

A sentence example of advice for the second type stored in the advice table 17 includes (i) an advice sentence example regarding to the range from the lower limit portion to the transition point and (ii) an advice sentence example regarding to the range from the transition point to the upper limit portion, and is illustrated in Table 2.

TABLE 2)
Sentence example of advice for second type

(i) A significant increase in your blood pressure is not recognized up to a
Na/K ratio of “BBB”.
You can eat with confidence up to the meal having the Na/K ratio of
“BBB”.
The meal having a Na/K ratio of “BBB” was of “⋄/⋄⋄” and “∇/∇∇”
(date).
(ii) Your blood pressure became 135 mmHg (the hypertension reference
value of the home blood pressure) when a Na/K ratio was “CCC”.
In order to keep your blood pressure within the reference value, you
should not take the meal having a Na/K ratio of “CCC” or more.
The meal having a Na/K ratio of “CCC” was of “□/□□” (date).

Numerical values of Na/K ratios (two significant digits) are applied to the fields of “BBB” and “CCC” by the processing (to be described later). In particular, a value according to a Na/K ratio coordinate of the transition point P2 is applied to the field of “BBB”. The numerical values of month/day are applied to the field of “⋄/⋄⋄”, the field of “∇/∇∇”, and the field of “□/□□”.

When the sensitivity type of the subject is the second type to illustrate the distribution of the data points in FIG. 4B, the x-coordinate (Na/K ratio coordinate) of the transition point P2 at which the first straight line L21 and the second straight line L22 are connected to each other is 2.5. The x coordinate (Na/K ratio coordinate) of the intersection point PX2 between the second straight line L22 and the hypertension reference value UL (=135 mmHg) is 3.5. Thus, the controller 11 reads the sentence example in Table 2 from the advice table 17, applies the value of 2.5 of the x-coordinate at the transition point P2 and the value of 3.5 of the x-coordinate at the intersection point PX2 to the fields of the “BBB” and “CCC” of the sentence example, respectively, and produces the advice in Table 2-A

TABLE 2-A
Advice for second type

(i) A significant increase in your blood pressure is not recognized up to a
Na/K ratio of 2.5.
You can eat with confidence up to the meal having a Na/K ratio of 2.5.
The meal having a Na/K ratio of 2.5 were of “5/09” and “5/10” (date).
(ii) Your blood pressure became 135 mmHg (the hypertension reference
value of the home blood pressure) when a Na/K ratio was 3.5.
In order to keep your blood pressure within the reference value, you
should not take the meal having a Na/K ratio of 3.5 or more.
The meal having a Na/K ratio of 3.5 was of 5/11 (date).

It is assumed that a sentence example of advice for the third type stored in the advice table 17 is such one shown in Table 3.

TABLE 3
Sentence example of advice for third type

(i) Your blood pressure became 135 mmHg (the hypertension reference
value of the home blood pressure) when a Na/K ratio was “DDD”.
In order to keep your blood pressure within the reference value, you
should not take the meal having a Na/K ratio of “DDD” or more.
The meal having a Na/K ratio of “DDD” was of “□/□□” (date).
(ii) You have a high influence on the blood pressure even if a Na/K ratio is
low.
Your blood pressure will be very high even if you have the low Na/K
ratio, so be careful of the meal so as not to increase your Na/K ratio as
much as possible.

At this point, a numerical value of Na/K ratio (two significant digits) is applied to the field of “DDD” by the processing (to be described later). In a field of “□/□□” column, a numerical value of month/day values is applied.

When the sensitivity type of the subject is the third type to illustrate the distribution of the data points in FIG. 4C, the x-coordinate (Na/K ratio coordinate) of an intersection point PX3 of the first straight line L31 and the hypertension reference value UL (=135 mmHg) is 3.0. The x-coordinate (Na/K ratio coordinate) of the transition point P3 where the first straight line L31 and the second straight line L32 are connected to each other exceeds the x-coordinate of 3.0 of the intersection point PX3. Thus, the controller 11 reads out the sentence example in Table 3 from the advice table 17, and applies the value of 3.0 of the x-coordinate at the intersection point PX3 to the field of the “DDD” of the sentence example to produce advice in Table 3-A.

TABLE 3-A
Advice for third type

(i) Your blood pressure became 135 mmHg (the hypertension reference
value of the home blood pressure) when a Na/K ratio was 3.0.
In order to keep your blood pressure within the reference value, you
should not take the meal having a Na/K ratio of 3.0 or more.
The meal having a Na/K ratio of 3.0 was of 5/11 (date).
(ii) You have a high influence on the blood pressure even if a Na/K ratio is
low.
Your blood pressure will be very high even if you have the low Na/K
ratio, so be careful of the meal so as not to increase your Na/K ratio as
much as possible.

It is assumed that a sentence example of advice for the fourth type stored in the advice table 17 is such one shown in Table 4.

TABLE 4
Sentence example of advice for fourth type

No correlation between your Na/K ratio and blood pressure is recognized.
A Na/K ratio does not seem to affect your blood pressure. It is expected
that other lifestyle habits such as exercise, sleep, and stress will greatly be
involved in a fluctuation in blood pressure.

When the sensitivity type of the subject is the fourth type to illustrate the distribution of the data points in FIG. 4D, the controller 11 reads out the sentence example of Table 4 from the advice table 17, and uses the sentence example as the advice as illustrated in Table 4-A.

TABLE 4-A
Advice for fourth type

No correlation between your Na/K ratio and blood pressure is recognized.
A Na/K ratio does not seem to affect your blood pressure. It is expected
that other lifestyle habits such as exercise, sleep, and stress will greatly be
involved in a fluctuation in blood pressure.

In this way, the advice can be created by a simple processing by applying the value according to the distribution of the data points to the advice sentence example stored in the advice table 17.

### (5) Output

Subsequently, in step S10 of FIG. 2, via the output unit 18, the controller 11 displays the sensitivity type of the subject obtained as the result of the type determination, the scatter diagram representing the distribution of the data points, and the advice according to the sensitivity type of the subject as the image on the display screen as information indicating the determination result.

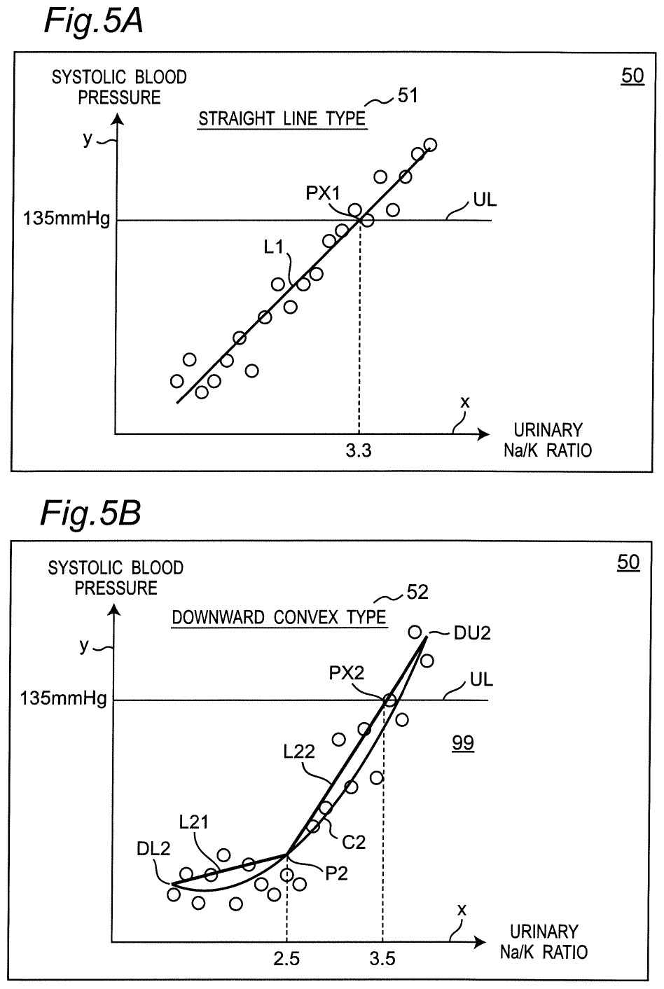

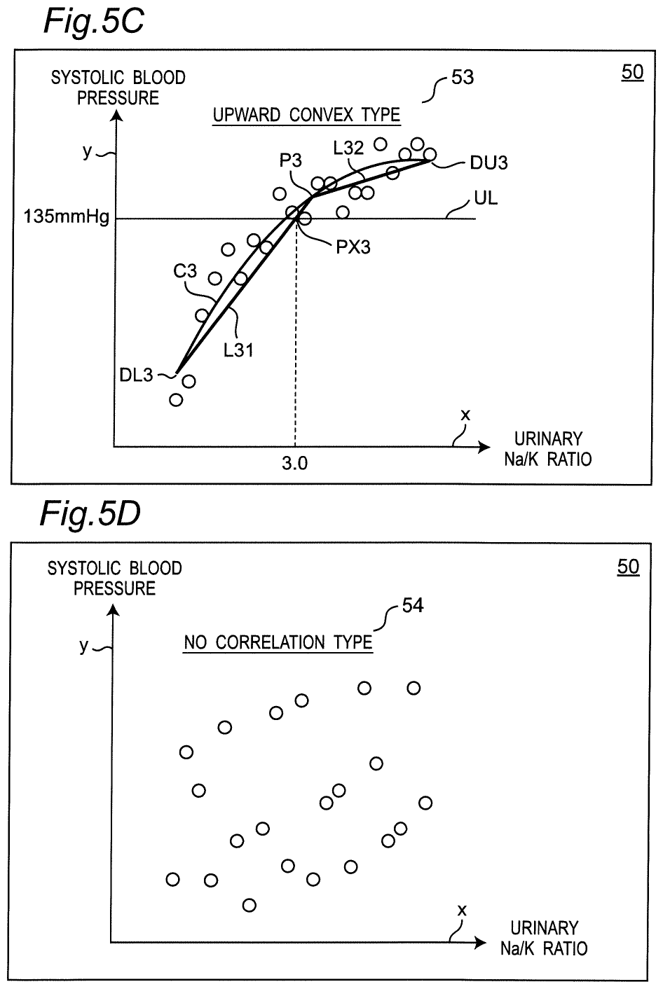

FIGS. 5A to 5D illustrate a mode in which the sensitivity type of the subject and the scatter diagram representing the distribution of the data points are displayed on a display screen 50.

FIG. 5A illustrates a display example when the sensitivity type of the subject is the first type. Although not illustrated for convenience, the display screen 50 also displays the advice in Table 1-A. In this display example, a character string 51 of “straight line type” is displayed in an upper portion of the display screen 50 as a name indicating that the sensitivity type of the subject is the first type. Below the character string 51, the scatter diagram representing the distribution of the data points in FIG. 4A is displayed together with the linear regression line L1 on the plane 99 formed by the horizontal axis (x-axis) representing the urinary Na/K ratio and the vertical axis (y-axis) representing the systolic blood pressure. Thus, the user can intuitively recognize that the sensitivity type of the subject is the first type (that is, the straight line type) by viewing the image. In this display example, that the x-coordinate (Na/K ratio coordinate) at the intersection point PX1 of the linear regression line L1 and the hypertension reference value UL (=135 mmHg) of the home blood pressure is 3.3 is displayed while the hypertension reference value UL (=135 mmHg) is displayed as the reference line parallel to the horizontal axis (x-axis). Thus, the user can intuitively recognize the Na/K ratio range where the blood pressure of the subject is lower than the hypertension reference value UL by viewing the image. After checking the content of the advice in Table 1-A, the user can provide the appropriate advice to the subject along the content of the advice in Table 1-A.

FIG. 5B illustrates a display example when the sensitivity type of the subject is the second type. Although not illustrated for convenience, the display screen 50 also displays the advice in Table 2-A. In this display example, a character string 52 of “downward convex type” is displayed in the upper portion of the display screen 50 as the name indicating that the sensitivity type of the subject is the second type. Below the character string 52, the scatter diagram representing the distribution of the data points in FIG. 4B is displayed together with the quadratic regression curve C2, the first straight line L21, and the second straight line L22 on the plane 99 formed by the horizontal axis (x-axis) representing the urinary Na/K ratio and the vertical axis (y-axis) representing the systolic blood pressure. Thus, the user can intuitively recognize that the sensitivity type of the subject is the second type (that is, the downward convex type) by viewing the image. In this display example, that the x-coordinate (Na/K ratio coordinate) at the intersection point PX2 of the second straight line L22 and the hypertension reference value UL (=135 mmHg) of the home blood pressure is 3.5 is displayed while the hypertension reference value UL (=135 mmHg) is displayed as the reference line parallel to the horizontal axis (x-axis). Thus, the user can intuitively recognize the Na/K ratio range where the systolic blood pressure of the subject is lower than the hypertension reference value UL by viewing the image. That the x-coordinate (Na/K ratio coordinate) at the transition point P2 between the first straight line L21 and the second straight line L22 is 2.5 is displayed in this display example. Thus, the user can intuitively recognize that the slope of the first straight line L21 and the slope of the second straight line L22 are different from each other before and after the transition point P2. That is, the user can intuitively recognize that the slope of the first straight line L21 from the lower limit portion DL2 to the transition point P2 is relatively small, and that the slope of the second straight line L22 from the transition point P2 to the upper limit portion DU2 is relatively large. After checking the content of the advice in Table 2-A, the user can provide the appropriate advice to the subject based on the content of the advice in Table 2-A. At this point, the advice in Table 2-A includes (i) advice about the range from the lower limit portion DL2 to the transition point P2 and (ii) advice about the range from the transition point P2 to the upper limit portion DU2, so that the user can provide the particularly appropriate advice to the subject.

FIG. 5C illustrates a display example when the sensitivity type of the subject is the third type. Although not illustrated for convenience, the display screen 50 also displays the advice illustrated in Table 3-A. In this display example, a character string 53 of “upward convex type” is displayed in the upper portion of the display screen 50 as the name indicating that the sensitivity type of the subject is the third type. Below the character string 53, the scatter diagram representing the distribution of the data points in FIG. 4C is displayed together with the quadratic regression curve C3, the first straight line L31, and the second straight line L32 on the plane 99 formed by the horizontal axis (x-axis) representing the urinary Na/K ratio and the vertical axis (y-axis) representing the systolic blood pressure. Thus, the user can intuitively recognize that the sensitivity type of the subject is the third type (that is, the upward convex type) by viewing the image. In this display example, that the x-coordinate (Na/K ratio coordinate) at the intersection point PX3 of the first straight line L31 and the hypertension reference value UL (=135 mmHg) of the home blood pressure is 3.0 is displayed while the hypertension reference value UL (=135 mmHg) is displayed as the reference line parallel to the horizontal axis (x-axis). Thus, the user can intuitively recognize the Na/K ratio range where the systolic blood pressure of the subject is lower than the hypertension reference value UL by viewing the image. The user can intuitively recognize that the slope of the first straight line L31 and the slope of the second straight line L32 are different from each other before and after the transition point P3 between the first straight line L31 and the second straight line L32. That is, the user can intuitively recognize that the slope of the first straight line L31 from the lower limit portion DL3 to the transition point P3 is relatively large, and that the slope of the second straight line L32 from the transition point P3 to the upper limit portion DU3 is relatively small. After checking the content of the advice in Table 3-A, the user can provide the appropriate advice to the subject based on the content of the advice in Table 3-A. At this point, the advice in Table 3-A corresponds to the fact that the slope of the first straight line L31 from the lower limit portion DL3 to the transition point P3 is relatively large and includes the advice that “(ii) You have a high influence on the blood pressure even if a Na/K ratio is low.”, so that the user can provide the particularly appropriate advice to the subject.

FIG. 5D illustrates a display example when the sensitivity type of the subject is the fourth type. Although not illustrated for convenience, the display screen 50 also displays the advice illustrated in Table 4-A. In this display example, a character string 54 of “no correlation type” is displayed in the upper portion of the display screen 50 as the name indicating that the sensitivity type of the subject is the fourth type. Below the character string 54, the scatter diagram representing the distribution of the data points in FIG. 4D is displayed on the plane 99 formed by the horizontal axis (x-axis) representing the urinary Na/K ratio and the vertical axis (y-axis) representing the systolic blood pressure. Thus, the user can intuitively recognize that the sensitivity type of the subject is the fourth type (that is, the no correlation type) by viewing the image. After checking the content of the advice in Table 4-A, the user can provide the appropriate advice to the subject based on the content of the advice in Table 4-A.

As described above, according to the method performed by the device 1 that evaluates the Na/K ratio sensitivity of the blood pressure, the sensitivity type of the subject can accurately be determined. Thus, the determination result can be reflected in guidance for a hypertension patient and the like, and the appropriate advice can be provided to the subject to improve the accuracy of the guidance.

### (First Modification)

In the above example, during the data input (step S1 in FIG. 2), the measured blood pressure data and the measured Na/K ratio data are associated with each other through a predetermined constant time difference (in the example, 12 hours). However, the present invention is not restricted to this configuration, but the time difference may be set for each subject.

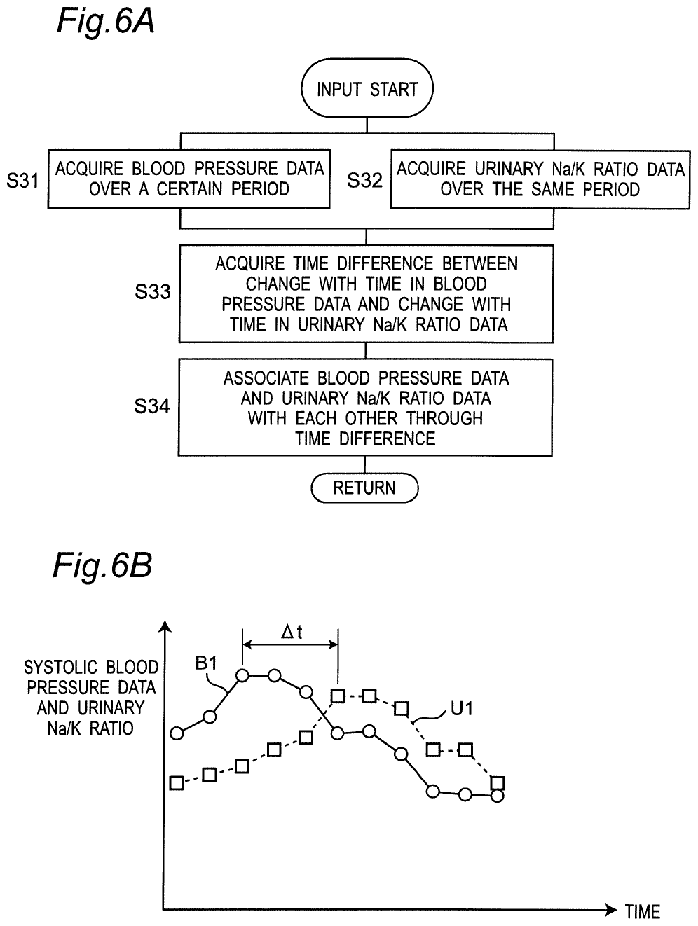

For example, as illustrated in step S31 of FIG. 6A, via the data input unit 12, the controller 11 measures the blood pressure data the plurality of times over a certain period of time with respect to a certain subject, and measures the urinary Na/K ratio data the plurality of times over the same period with respect to the subject as illustrated in step S32. At this point, as illustrated in FIG. 6B, the measured blood pressure data and the measured Na/K ratio data usually change with time by the influence of the Na/K ratio in the intake food. In FIG. 6B, the measured blood pressure data for each time is represented by the mark “0”, and the change with time in the blood pressure data is represented by a line graph B 1. The measured urinary Na/K ratio data for each time is represented by the mark “□”, and the change with time in the urinary Na/K ratio data is represented by a line graph U1. In the example of FIG. 6B, the blood pressure data indicates a maximum from the third measurement to the fifth measurement, and the urinary Na/K ratio data indicates the maximum from the sixth measurement to the eighth measurement after a time difference Δt.

As illustrated in step S33 of FIG. 6A, the controller 11 acts as a time difference acquisition unit to acquire the time difference Δt between the change with time in the measured blood pressure data and the change with time in the measured Na/K ratio data with respect to the subject. In this example, it is assumed that the time difference Δt is 11 hours.

As illustrated in step S34, the controller 11 acts as a data association unit to associate the measured blood pressure data and the measured Na/K ratio data are correlated with each other through the time difference Δt (in this example, 11 hours). Consequently, the measured blood pressure data and the measured Na/K ratio data can accurately be associated with each other for each subject.

### (Second Modification)

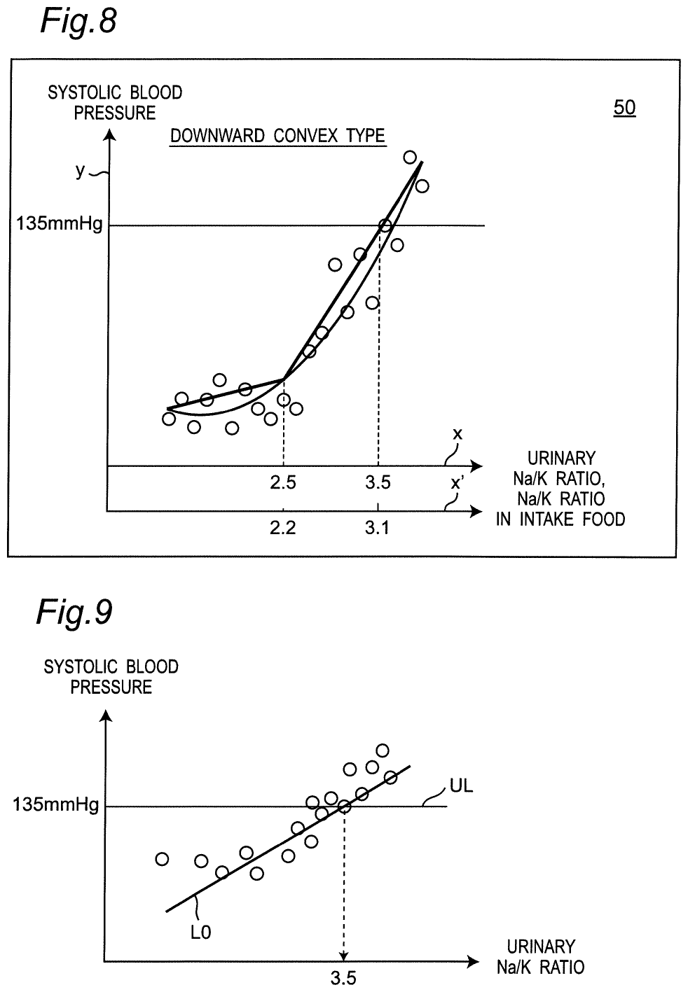

In the above embodiment, during the output (step S10 in FIG. 2), as illustrated in FIGS. 5A to 5D, a scale representing the urinary Na/K ratio was attached to the horizontal axis (x-axis) of the scatter diagram displayed on the display screen 50. However, the present invention is not limited to the embodiment. For example, as illustrated in FIG. 8, in the scatter diagram of FIG. 5B, a horizontal axis (x′-axis) with a scale indicating the Na/K ratio in the intake food by conversion based on a knowledge that the ratios of the amounts of sodium and potassium taken in by the human through the meal and excreted in the urine are 86% and 77%, respectively (Non-patent Literature 2) may be added and displayed in parallel. In this example, the urinary Na/K ratios of 3.5 and 2.5 are converted into the Na/K ratios of 3.1 and 2.2 in the intake food, respectively. In such a case, the user can more easily guide the subject to limit the Na/K ratio in the intake food.

### (Third Modification)

In the above embodiment, the device 1 includes the sphygmomanometer 30 and the Na/K meter 31. However, the device 1 is not limited to this configuration. For example, the device 1 includes a communication unit (not illustrated) that can communicate with an external network in a wired or wireless manner, and the blood pressure data and the urinary Na/K ratio data that are measured several times over a certain period with respect to each subject may be input from the external network while associated with each other. In that case, as in the above example, the sensitivity type of the subject can be determined, and therefore the accuracy of the guidance for the hypertension patients and the like can be increased.

The method for evaluating the Na/K ratio sensitivity of the blood pressure may be recorded in a non-transitory recording medium such as CD (compact disc) or DVD (digital universal disc), and EEPROM (electrically rewritable non-volatile memory) as a computer program. In such a case, by causing a substantial computer device such as a personal computer, a smartphone or the like to read the program recorded in the recording medium, it is possible to make the computer device perform the method.

In the above embodiment, the correlation between the systolic blood pressure and the urinary Na/K ratio is evaluated as the Na/K ratio sensitivity of the blood pressure. However, the present invention is not limited to the embodiment. The present invention can also be applied to the correlation between a diastolic blood pressure (DBP) and the urinary Na/K ratio, and the correlation between a pulse pressure and the urinary Na/K ratio.

As is described above, a device of the present disclosure is a device that evaluates Na/K ratio sensitivity of a blood pressure, the device comprising:

a data input unit that inputs blood pressure data and urinary Na/K ratio data measured a plurality of times over a certain period with respect to a certain subject while associating the blood pressure data and the urinary Na/K ratio data with each other;

a type determination unit that produces a scatter diagram representing data points decided by the Na/K ratio data and the blood pressure data associated with each other on a plane formed by a first coordinate axis representing a Na/K ratio and a second coordinate axis representing a blood pressure, and determines sensitivity type of the subject as one of predetermined four types existing actually according to a distribution of the data points in the scatter diagram; and an output unit that outputs information representing a determination result of the type determination unit.

Typically, the “first coordinate axis” is the horizontal axis (x-axis) and the “second coordinate axis” is the vertical axis (y-axis), and may be reversed.

“Information indicating the determination result” may widely include information relating to the Na/K ratio sensitivity of the blood pressure, such as a name indicating the determined sensitivity type, the image representing the scatter diagram, and advice according to each of the four types.

In the device of the present disclosure, in order to evaluate the Na/K ratio sensitivity of the blood pressure, a data input unit inputs blood pressure data and urinary Na/K ratio data measured a plurality of times over a certain period with respect to a certain subject while associating the blood pressure data and the urinary Na/K ratio data with each other. A type determination unit produces a scatter diagram representing data points decided by the Na/K ratio data and the blood pressure data associated with each other on a plane formed by a first coordinate axis representing a Na/K ratio and a second coordinate axis representing a blood pressure, and determines sensitivity type of the subject as one of predetermined four types existing actually according to a distribution of the data points in the scatter diagram. An output unit outputs information representing a determination result of the type determination unit. In this way, according to the device of the present disclosure, the sensitivity type of the subject can be determined. Thus, when the determination result is reflected in guidance for the hypertension patient for example, the accuracy of the guidance is increased.

In the device of one embodiment,

the four types are

a first type in which, on the plane, the blood pressure with respect to the Na/K ratio changes along one straight line over a range from a lower limit portion to an upper limit portion where the data points are distributed,
a second type in which, on the plane, the blood pressure with respect to the Na/K ratio changes over the range from a lower limit portion to an upper limit portion where the data points are distributed while being curved more convexly than one straight line connecting the lower limit portion and the upper limit portion with respect to the first coordinate axis,
a third type in which, on the plane, the blood pressure with respect to the Na/K ratio changes over the range from a lower limit portion to an upper limit portion where the data points are distributed while being curved more concavely than one straight line connecting the lower limit portion and the upper limit portion with respect to the first coordinate axis, and
a fourth type having no correlation between the Na/K ratio and the blood pressure.

In the description, the “lower limit portion” of the distribution is an end portion on the side where both the Na/K ratio and the blood pressure are small on the assumption that the blood pressure changes with a positive correlation with respect to the Na/K ratio. Similarly, the “upper limit portion” of the distribution is an end portion on the side where both the Na/K ratio and the blood pressure are large on the assumption that the blood pressure changes with a positive correlation with respect to the Na/K ratio. The “end portion” is not limited to a point, but may be a certain region.

As described above, the first type to the fourth type are sensitivity types existing actually. Thus, according to the device of one embodiment, the sensitivity type of the subject can be determined as any one of the sensitivity types existing actually.

In the device of one embodiment,

the type determination unit
performs regression analysis on the distribution using a quadratic function to obtain a quadratic regression curve and a p-value indicating significance, and determines whether the sensitivity type of the subject belongs to a group of the second type and the third type or a group of the first type and the fourth type based on whether the p-value is less than a predetermined first threshold,
determines whether the sensitivity type of the subject is the second type or the third type according to a sign of a coefficient of a second-order term of the quadratic regression curve when the sensitivity type of the subject belongs to the group of the second type and the third type, and
further performs the regression analysis on the distribution using a linear function to obtain a linear regression line and a p-value indicating significance when the sensitivity type of the subject belongs to the group of the first type and the fourth type, and determining whether the sensitivity type of the subject is the first type or the fourth type based on whether the p-value is less than a predetermined second threshold.

The “first threshold” is typically set to 0.05. Similarly, the “second threshold” is typically set to 0.05.

In the device of one embodiment, the type determination unit performs regression analysis on the distribution using a quadratic function to obtain a quadratic regression curve and a p-value indicating significance, and determines whether the sensitivity type of the subject belongs to a group of the second type and the third type or a group of the first type and the fourth type based on whether the p-value is less than a predetermined first threshold. Subsequently, the type determination unit determines whether the sensitivity type of the subject is the second type or the third type according to a sign of a coefficient of a second-order term of the quadratic regression curve when the sensitivity type of the subject belongs to the group of the second type and the third type. On the other hand, the type determination unit further performs the regression analysis on the distribution using a linear function to obtain a linear regression line and the p-value indicating the significance when the sensitivity type of the subject belongs to the group of the first type and the fourth type, and determining whether the sensitivity type of the subject is the first type or the fourth type based on whether the p-value is less than a predetermined second threshold. According to the device of one embodiment, the sensitivity type of the subject can accurately be determined by simple processing.

In the device of one embodiment, the data input unit associates the measured blood pressure data with the measured Na/K ratio data through a predetermined constant time difference.

In general, an influence of sodium and potassium taken in by a human through a meal appears first in the blood pressure, and appears in the urinary Na/K ratio approximately half a day later (12 hours). For this reason, in the device of one embodiment, the data input unit associates the measured blood pressure data with the measured Na/K ratio data through a predetermined constant time difference (typically, 12 hours). Thus, the measured blood pressure data and the measured Na/K ratio data can easily be input while associated with each other.

The device of one embodiment further comprises:

a time difference acquisition unit that acquires a time difference between a change with time in the measured blood pressure data and a change with time in the measured Na/K ratio data with respect to the subject; and

a data association unit that associates the measured blood pressure data with the measured Na/K ratio data through the time difference.

In the device of one embodiment, the time difference acquisition unit acquires the time difference between a change with time in the measured blood pressure data and a change with time in the measured Na/K ratio data with respect to the subject. The data association unit associates the measured blood pressure data with the measured Na/K ratio data through the time difference. Thus, the measured blood pressure data and the measured Na/K ratio data can accurately be associated with each other for each subject.

In the device of one embodiment, the output unit outputs an image representing the scatter diagram.

In the description, the term “output an image” widely includes displaying the image on the display screen, printing the image on paper, and storing non-transiently data representing the image in a recording medium such as a memory.

In the device of one embodiment, the output unit outputs an image representing the scatter diagram. Thus, a user (typically, a medical worker such as a doctor and a nurse) can intuitively recognize which one of the four types the sensitivity type of the subject belongs to by viewing the image.

The device of one embodiment further comprises a two-line approximation unit that obtains a first straight line and a second straight line, which pass through the lower limit portion, the upper limit portion, respectively, and are connected and bent to each other at a certain transition point along the quadratic regression curve so as to approximate the quadratic regression curve on the plane, when the sensitivity type of the subject is the second type or the third type.

At this point, for example, the point at which the quadratic regression curve is separated farthest from the one straight line between the lower limit portion and the upper limit portion can be adopted as the “certain transition point” on the quadratic regression curve.

In the device of one embodiment, a two-line approximation unit obtains a first straight line and a second straight line, which pass through the lower limit portion, the upper limit portion, respectively, and are connected and bent to each other at a certain transition point along the quadratic regression curve so as to approximate the quadratic regression curve on the plane, when the sensitivity type of the subject is the second type or the third type. Thus, the range from the lower limit portion to the transition point can be approximated by the first straight line, and the range from the transition point to the upper limit portion can be approximated by the second straight line. Consequently, the accuracy of the approximation can be improved as compared with the case where the correlation between the blood pressure and the urinary Na/K ratio is simply approximated by one straight line. The user easily recognizes a slope of the correlation between the blood pressure and the urinary Na/K ratio while dividing the slope of the correlation between the blood pressure and the urinary Na/K ratio in the range from the lower limit portion to the transition point and the range from the transition point to the upper limit portion.

In the device of one embodiment, the output unit outputs an image representing the first and second straight lines on the plane when the sensitivity type of the subject is the second type or the third type.

In the device of one embodiment, the output unit outputs an image representing the first and second straight lines on the plane when the sensitivity type of the subject is the second type or the third type. Thus, the user can view the image to intuitively recognize that the slope of the first straight line and the slope of the second straight line are different from each other before and after the transition point. That is, when the sensitivity type of the subject is the second type, the user can intuitively recognize that the slope of the first straight line from the lower limit portion to the transition point is relatively small, and that the slope of the second straight line from the transition point to the upper limit portion is relatively large. On the other hand, when the sensitivity type of the subject is the third type, the user can intuitively recognize that the slope of the first straight line from the lower limit portion to the transition point is relatively large, and that the slope of the second straight line from the transition point to the upper limit portion is relatively small.

In the device of one embodiment, the output unit outputs an image representing a reference line of the blood pressure on the plane.

In the description, the “reference line” of the blood pressure means a line representing the blood pressure reference (including the hypertension reference of 135 mmHg and 85 mmHg at home) according to the “Guidelines for the Management of Hypertension 2014” by the Japanese Society of Hypertension, for example. For example, the “reference line” may be a classification published by the World Health Organization (WHO)/International Society of Hypertension (ISH) and a classification published by the American Joint Committee on Hypertension (JNC)/American Heart Association (AHA).

In the device of one embodiment, the output unit outputs an image representing a reference line of the blood pressure on the plane. Thus, when the user can intuitively recognize the Na/K ratio range in which the blood pressure of the subject is lower than the reference line by viewing the image.

In the device of one embodiment, the output unit outputs advice according to the sensitivity type of the subject among the four types.

In the device of one embodiment, the output unit outputs advice according to the sensitivity type of the subject among the four types. Consequently, the appropriate advice can be provided to the subject.

The device of one embodiment further comprises:

an advice table in which sentence examples of advice according to the four types are stored, at least one of the sentence examples of the advice including a field to which a value according to the distribution of the data points should be applied; and

an advice production unit that reads the sentence example according to the sensitivity type of the subject from the advice table, and produces advice by applying a value according to the distribution of the data points to the field when the sentence example includes the field.

The device of one embodiment includes an advice table in which sentence examples of advice according to the four types are stored, at least one of the sentence examples of the advice including a field to which a value according to the distribution of the data points should be applied. An advice production unit reads the sentence example according to the sensitivity type of the subject from the advice table, and produces advice by applying a value according to the distribution of the data points to the field when the sentence example includes the field. Thus, the advice according to the distribution of the data points can be produced through simple processing. Consequently, the appropriate advice can be provided to the subject.

The device of one embodiment further comprises:

an advice table in which sentence examples of advice according to the four types are stored, the sentence examples of the second type including a field to which a value according to the transition point should be applied; and

an advice production unit that reads the sentence example of the second type from the advice table when the sensitivity type of the subject is the second type, and produces advice by applying a value according to the transition point to the field of the sentence example.

The device of one embodiment includes an advice table in which sentence examples of advice according to the four types are stored, the sentence examples of the second type including a field to which a value according to the transition point should be applied. An advice production unit reads the sentence example of the second type from the advice table when the sensitivity type of the subject is the second type, and produces advice by applying a value according to the transition point to the field of the sentence example. Thus, the advice according to the transition point can be produced through simple processing. Consequently, the particularly appropriate advice can be provided to the subject whose sensitivity type is the second type.

The device of one embodiment further comprises:

a sphygmomanometer that measures the blood pressure data; and

a measuring instrument that measures the urinary Na/K ratio data.

In the device of one embodiment, the blood pressure data is measured by the sphygmomanometer. The urinary Na/K ratio data is measured by the measuring instrument. Thus, the blood pressure data and the urinary Na/K ratio data are easily measured a plurality of times over a certain period.

Desirably, the input of the blood pressure data from the sphygmomanometer by the data input unit and the input of the urinary Na/K ratio data from the measuring instrument by the data input unit are performed by wired or wireless communication.

In another aspect, a method of the present disclosure is a method for assessing Na/K ratio sensitivity of a blood pressure, the method comprising:

inputting blood pressure data and urinary Na/K ratio data measured a plurality of times over a certain period with respect to a certain subject while associating the blood pressure data and the urinary Na/K ratio data with each other;

producing a scatter diagram representing data points decided by the Na/K ratio data and the blood pressure data associated with each other on a plane formed by a first coordinate axis representing a Na/K ratio and a second coordinate axis representing a blood pressure, and determining sensitivity type of the subject as one of predetermined four types existing actually according to a distribution of data points in the scatter diagram; and

outputting information representing a determination result.

According to the method of the present disclosure, the sensitivity type of the subject can be determined. Thus, when the determination result is reflected in guidance for the hypertension patient for example, the accuracy of the guidance is increased.

In another aspect, a program of the present disclosure is a program causing a computer to perform the method.

According to the program of the present disclosure, the method can be performed by a computer.

As is clear from the above, according to the device and the method for evaluating the Na/K ratio sensitivity of the blood pressure of the present disclosure, the sensitivity type of the subject can be determined. According to the program of the present disclosure, the method can be performed by the computer.

The above embodiments are illustrative, and are modifiable in a variety of ways without departing from the scope of this invention. It is to be noted that the various embodiments described above can be appreciated individually within each embodiment, but the embodiments can be combined together. It is also to be noted that the various features in different embodiments can be appreciated individually by its own, but the features in different embodiments can be combined.

1. A device that evaluates Na/K ratio sensitivity of a blood pressure, the device comprising:
a data input unit that inputs blood pressure data and urinary Na/K ratio data measured a plurality of times over a certain period with respect to a certain subject while associating the blood pressure data and the urinary Na/K ratio data with each other;
a type determination unit that produces a scatter diagram representing data points decided by the Na/K ratio data and the blood pressure data associated with each other on a plane formed by a first coordinate axis representing a Na/K ratio and a second coordinate axis representing a blood pressure, and determines sensitivity type of the subject as one of predetermined four types existing actually according to a distribution of the data points in the scatter diagram; and
an output unit that outputs information representing a determination result of the type determination unit.

2. The device according to claim 1, wherein
the four types are
a first type in which, on the plane, the blood pressure with respect to the Na/K ratio changes along one straight line over a range from a lower limit portion to an upper limit portion where the data points are distributed,
a second type in which, on the plane, the blood pressure with respect to the Na/K ratio changes over the range from a lower limit portion to an upper limit portion where the data points are distributed while being curved more convexly than one straight line connecting the lower limit portion and the upper limit portion with respect to the first coordinate axis,
a third type in which, on the plane, the blood pressure with respect to the Na/K ratio changes over the range from a lower limit portion to an upper limit portion where the data points are distributed while being curved more concavely than one straight line connecting the lower limit portion and the upper limit portion with respect to the first coordinate axis, and
a fourth type having no correlation between the Na/K ratio and the blood pressure.

3. The device according to claim 2, wherein
the type determination unit
performs regression analysis on the distribution using a quadratic function to obtain a quadratic regression curve and a p-value indicating significance, and determines whether the sensitivity type of the subject belongs to a group of the second type and the third type or a group of the first type and the fourth type based on whether the p-value is less than a predetermined first threshold,
determines whether the sensitivity type of the subject is the second type or the third type according to a sign of a coefficient of a second-order term of the quadratic regression curve when the sensitivity type of the subject belongs to the group of the second type and the third type, and
further performs the regression analysis on the distribution using a linear function to obtain a linear regression line and a p-value indicating significance when the sensitivity type of the subject belongs to the group of the first type and the fourth type, and determining whether the sensitivity type of the subject is the first type or the fourth type based on whether the p-value is less than a predetermined second threshold.

4. The device according to claim 1, wherein the data input unit associates the measured blood pressure data with the measured Na/K ratio data through a predetermined constant time difference.

5. The device according to claim 1, further comprising:
a time difference acquisition unit that acquires a time difference between a change with time in the measured blood pressure data and a change with time in the measured Na/K ratio data with respect to the subject; and
a data association unit that associates the measured blood pressure data with the measured Na/K ratio data through the time difference.

6. The device according to claim 1, wherein the output unit outputs an image representing the scatter diagram.

7. The device according to claim 3, further comprising a two-line approximation unit that obtains a first straight line and a second straight line, which pass through the lower limit portion, the upper limit portion, respectively, and are connected and bent to each other at a certain transition point along the quadratic regression curve so as to approximate the quadratic regression curve on the plane, when the sensitivity type of the subject is the second type or the third type.

8. The device according to claim 7, wherein the output unit outputs an image representing the first and second straight lines on the plane when the sensitivity type of the subject is the second type or the third type.

9. The device according to claim 6, wherein the output unit outputs an image representing a reference line of the blood pressure on the plane.

10. The device according to claim 1, wherein the output unit outputs advice according to the sensitivity type of the subject among the four types.

11. The device according to claim 10, further comprising:
an advice table in which sentence examples of advice according to the four types are stored, at least one of the sentence examples of the advice including a field to which a value according to the distribution of the data points should be applied; and
an advice production unit that reads the sentence example according to the sensitivity type of the subject from the advice table, and produces advice by applying a value according to the distribution of the data points to the field when the sentence example includes the field.

12. The device according to claim 7, further comprising:
an advice table in which sentence examples of advice according to the four types are stored, the sentence examples of the second type including a field to which a value according to the transition point should be applied; and
an advice production unit that reads the sentence example of the second type from the advice table when the sensitivity type of the subject is the second type, and produces advice by applying a value according to the transition point to the field of the sentence example.

13. The device according to claim 1, further comprising:
a sphygmomanometer that measures the blood pressure data; and
a measuring instrument that measures the urinary Na/K ratio data.

14. A method for assessing Na/K ratio sensitivity of a blood pressure, the method comprising:
inputting blood pressure data and urinary Na/K ratio data measured a plurality of times over a certain period with respect to a certain subject while associating the blood pressure data and the urinary Na/K ratio data with each other;
producing a scatter diagram representing data points decided by the Na/K ratio data and the blood pressure data associated with each other on a plane formed by a first coordinate axis representing a Na/K ratio and a second coordinate axis representing a blood pressure, and determining sensitivity type of the subject as one of predetermined four types existing actually according to a distribution of data points in the scatter diagram; and
outputting information representing a determination result.

15. A program causing a computer to perform the method according to claim 14.
